(19) United States
(12) Patent Application Publication
(10) Pub. No.: US 2014/0328851 A1
(43) Pub. Date: Nov. 6, 2014

(54) Cholesterol Ester Transfer Protein (CETP) Inhibitor Polypeptide Antibodies for Prophylactic and Therapeutic Anti-Atherosclerosis Treatments

(71) Applicant: The Regents of the University of California, Oakland, CA (US)

(72) Inventors: Gang Ren, El Cerrito, CA (US); Lei Zhang, Albany, CA (US)

(73) Assignee: THE REGENTS OF THE UNIVERSITY OF CALIFORNIA, Oakland, CA (US)

(21) Appl. No.: 14/279,182

(22) Filed: May 15, 2014

Related U.S. Application Data
(63) Continuation of application No. PCT/US2012/065697, filed on Nov. 16, 2012.
(60) Provisional application No. 61/560,751, filed on Nov. 16, 2011.

(51) Int. Cl. C07K 16/18; A61K 49/00

(52) U.S. Cl. CPC C07K 16/18; A61K 49/0004
USPC 424/139.1; 530/387.9; 536/23.53; 530/387.3; 530/391.3; 424/185.1; 435/320.1; 435/331; 435/252.33; 435/69.6

(57) ABSTRACT

Herein are described two antibodies that can inhibit CETP-lipoproteins interaction and CETP activity. Presently described are an antibody or fragment thereof capable of specifically binding to an epitope of the N-terminal or C-terminal domains of CETP and methods of using these antibodies for separation, identification, diagnosis and therapy.

### CROSS-REFERENCE TO RELATED APPLICATIONS

The present application claims priority to International Patent Application No. PCT/US2012/065697, filed on Nov. 16, 2012, which claims priority to U.S. Provisional Patent Application No. 61/560,751, filed on Nov. 16, 2011, the entirety of both of which are hereby incorporated by reference.

### STATEMENT OF GOVERNMENTAL SUPPORT

The invention was made with government support under Contract No. DE-AC02-05CH11231 awarded by the U.S. Department of Energy under. The government has certain rights in the invention.

### BACKGROUND OF THE INVENTION

1. Field of the Invention

The present invention is in the field of antibodies and in some embodiments, treatments for artherosclerosis and cardiovascular diseases.

2. Related Art

Cholesteryl ester transfer protein (CETP) mediates the transfer of neutral lipids, including cholesteryl esters (CEs) and triglycerides (TGs), between high-density lipoproteins (HDL), low-density lipoproteins (LDL) and very low-density lipoproteins (VLDL) (Barter, P. J. et al. Cholesteryl ester transfer protein: a novel target for raising HDL and inhibiting atherosclerosis. Arterioscler Thromb Vasc Biol 23, 160-7 (2003)). Lipoprotein particles contain a neutral lipid core composed of CE and TG surrounded by a surface monolayer of phospholipids (PL), free cholesterol (FC), and apolipoproteins, most notably, apo B-100 in LDL and VLDL and apo A-I in HDL. An elevated level of LDL-cholesterol (LDL-C) and/or a low level of HDL-cholesterol (HDL-C) in human plasma are major risk factors for cardiovascular disease (CVD) (Camejo, G., Waich, S., Quintero, G., Berrizbeitia, M. L. & Lalaguna, F. The affinity of low density lipoproteins for an arterial macromolecular complex. A study in ischemic heart disease and controls. Atherosclerosis 24, 341-54 (1976); Gordon, T., Castelli, W. P., Hjortland, M. C., Kannel, W. B. & Dawber, T. R. High density lipoprotein as a protective factor against coronary heart disease. The Framingham Study. Am J Med 62, 707-14 (1977)). Increased CETP can reduce HDL-C concentration (See Hayek, T. et al. Hypertriglyceridemia and cholesteryl ester transfer protein interact to dramatically alter high density lipoprotein levels, particle sizes, and metabolism. Studies in transgenic mice. J Clin Invest 92, 1143-52 (1993)) and CETP deficiency is associated with elevated HDL-C levels (Brown, M. L. et al. Molecular basis of lipid transfer protein deficiency in a family with increased high-density lipoproteins. Nature 342, 448-51 (1989); Inazu, A. et al. Increased high-density lipoprotein levels caused by a common cholesteryl-ester transfer protein gene mutation. N Engl J Med 323, 1234-8 (1990)) Inhibition of CETP raises HDL cholesterol and lowers LDL, and has been actively pursued by pharmaceutical companies as a novel approach to treat cardiovascular disease. CETP inhibitors, including torcetrapib, anacetrapib and dalcetrapib have been investigated in clinical trials for treating CVD (Niesor, E. J. Different effects of compounds decreasing cholesteryl ester transfer protein activity on lipoprotein metabolism. Curr Opin Lipidol (2011); Miyares, M. A. Anacetrapib and dalcetrapib: two novel cholesteryl ester transfer protein inhibitors. Ann Pharmacother 45, 84-94 (2011); Kappelle, P. J., van Tol, A., Wolffenbuttel, B. H. & Dullaart, R. P. Cholesteryl Ester Transfer Protein Inhibition in Cardiovascular Risk Management: Ongoing Trials will End the Confusion. Cardiovasc Ther (2011)). Despite the intense clinical interest in CETP inhibition, little is known concerning the molecular mechanisms of CETP-mediated lipid transfer among lipoproteins, or even how CETP interacts with lipoproteins.

CETP is a hydrophobic glycoprotein of 476 amino acids (˜53 kDa, before posttranslational modification). The recently solved crystal structure, which CETP transfers HDL-cholesteryl esters (CEs) and VLDL-TG, has remained elusive of CETP reveals a central tunnel within a roughly boomerang shaped protein molecule (Qiu et al. 2007). Its crystal structure reveals a banana-shaped molecule with N- and C-terminal β-barrel domains, a central β-sheet, and a ˜60 Å-long hydrophobic central cavity. The cavity, which can accommodate two CE molecules, communicates with two pores near the central β-sheet domain. These pores, occupied by two PL molecules, could be gates for the interaction of the central cavity with the aqueous environment or lipoproteins. (Qiu, X. et al. Crystal structure of cholesteryl ester transfer protein reveals a long tunnel and four bound lipid molecules. Nat Struct Mol Biol 14, 106-13 (2007)).

In spite of this new knowledge, the mechanism by which lipid transfer occurs is not known. Three CETP neutral lipid transfer hypotheses were proposed two decades ago: 1) a shuttle mechanism that involves CETP collecting CEs from one lipoprotein and delivering them through the aqueous phase to another lipoprotein (Barter, P. J. & Jones, M. E. Kinetic studies of the transfer of esterified cholesterol between human plasma low and high density lipoproteins. J Lipid Res 21, 238-49 (1980)); 2) a tunnel mechanism in which CETP bridges two lipoproteins forming a ternary complex, with lipids flowing from the donor to acceptor lipoprotein through the CETP molecule (Ihm, J., Quinn, D. M., Busch, S. J., Chataing, B. & Harmony, J. A. Kinetics of plasma protein-catalyzed exchange of phosphatidylcholine and cholesteryl ester between plasma lipoproteins. J Lipid Res 23, 1328-41 (1982)); and 3) a modified tunnel mechanism implicating a CETP dimer. (Tall, A. R. Plasma cholesteryl ester transfer protein. J Lipid Res 34, 1255-74 (1993).

Monoclonal antibodies to full-length human CETP are described by Kamada et al, in U.S. Pat. Nos. 6,410,020 and 6,140,474. Rittershaus et al also describe modulation of CETP activity in U.S. Pat. No. 7,078,036, using CETP amino acids 16 to 31, linked to amino acids 349 to 367 and amino acids 461 to 476 of the amino acid sequence for mature human CETP. Rittershaus also teach a multivalent vaccine peptide containing B cell epitopes from the homologous regions of the rabbit CETP (i.e., amino acids 350 to 368 and 481 to 496). Our present studies show that the epitope that should be targeted to modulate CETP activity effectively is found in a different region of CETP. This has been validated by the lack of progress in previous approaches.

### SUMMARY OF THE INVENTION

The present invention provides for an antibody or fragment thereof capable of specifically binding to an epitope of the CETP amino acid sequence 44-61: ITGEKAMMLLGQVKYGLH (SEQ ID NO:1); 95-116: GTLKYGYTTAWWLGIDQSIDFE (SEQ ID NO:2); 151-171: LLHLQGEREPGWIKQLFTNF (SEQ ID NO:3); 98-112: KYGYTTAWWLGIDQS (SEQ ID NO:4); 288-360: GRLMLSLMGDEFKAVLETWGFNTNQEIFQEVVGGFPSQA (SEQ ID NO:5), 349-360: FLFPRPDQQHSVA (SEQ ID NO:6), or 101-110: YTTAWWLGID (SEQ ID NO:7) or a fragment of at least 5, 6, or 7 amino acids thereof.

The present invention relates to a polynucleotide encoding the antibody or fragment thereof of the present invention, vectors comprising said polynucleotide as well as cells comprising the afore-mentioned polynucleotide or vector. The present invention also provides a method for preparing antibodies capable of binding to an epitope of a peptide derived from one amino acid sequence selected from the group of SEQ ID NOS:1-7.

The present invention provides for a hybridoma capable of producing an antibody or fragment thereof of the present invention.

The present invention provides for a method of isolating a peptide of interest, comprising: (a) contacting (i) a peptide of interest derived from amino acid sequences SEQ ID NO:1-7 or a fragment thereof, and (ii) the antibody or fragment thereof of the present invention, and (b) separating at least a partial population of the antibody or fragment thereof, and any bound molecule thereto, from molecules not bound to the antibody or fragment thereof.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1 provides images showing structural conformations of CETP bound to LDL or VLDL and CETP interactions between lipoproteins by optimized negative-staining EM. (A) Linear or banana-shaped CETPs (˜100±10 Å long) associated with the surfaces of LDL particles, and (B) linear-shaped CETPs (˜105±10 Å long, red arrows) protruding from the surfaces of VLDL particles. (C) linear-shaped CETPs (˜25-55 Å long) bridge HDL particles (diameter ˜85-110 Å) to LDL particles (diameter ˜200-270 Å), forming ternary complexes. (D) VLDL particles (diameter ˜370-570 Å) connected to HDL particles (diameter ˜85-110 Å) via linear-shaped CETPs (˜35-65 Å long) forming ternary complexes similar to that of LDL. Bars=100 Å.

FIG. 2 provides images and a graph showing analysis of HDL size change during incubation with CETP. Samples were collected and viewed by NS-EM. (A) When HDL is incubated with LDL, but without CETP, the HDL particle size remains unchanged up to 4 hours. (B) When HDL is incubated with CETP and LDL, the HDL particle size decreases after 1 hour of incubation (bar=300 Å). (C) A total of ˜500 HDL particles for each incubation protocol was selected from NS-EM micrographs for quantitative size analysis. The geometric sizes of particles were calculated and expressed as the mean±SD. Quantifying the size of HDL particles for HDL alone (diamond, ⋄), HDL/LDL (square, □) and HDL/CETP (triangle, ▴) show no size changes during the incubation time except for the ternary mixture of HDL/CETP/LDL (circle). The diameter of HDL particles following incubations with LDL and CETP in the presence of antibodies H300 (triangle facing left, [symbol] ) and N13 (triangle facing right, [symbol] ) are also shown.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

Using electron microscopy, we have identified the CETP end that interacts with HDL and thereby we also reduced the end for LDL/VLDL interactions. Human CETP (cholesteryl ester transfer protein precursor) protein sequence and information is described in GenBank Accession No. NP_{—}000069.2 GI:169636439, hereby incorporated by reference. The human CETP protein sequence is also identified herein as SEQ ID NO: 8) The studies described in the Examples reveal that CETP binds HDL through the N-terminal tip of the boomerang structure, in a general area identified as amino acids (herein also referred to as “loops”) 44-61 (ITGEKAMMLLGQVKYGLH; SEQ ID NO:1), 95-116 (GTLKYGYTTAWWLGIDQSIDFE; SEQ ID NO:2), and 151-171 (LLHLQGEREPGWIKQLFTNFI; SEQ ID NO:3). A small β-sheet identified by four alternatively charged strands (D42-E46, K56-H60, K94-K98, D114-E115) may also be involved in the binding. This is markedly different from the previously established hypothesis that the concave surface of CETP, including the C-terminal 26 amino acid helix, was the key for lipoprotein binding. Most notably, W105-W106, at the very tip of the 98-112 loop (KYGYTTAWWLGIDQS; SEQ ID NO:4) of the CETP molecule, is described herein as providing an excellent peptide epitope for various immunology approaches, such as for generating antibody (or their permutations, fragments, chimeras, or other engineered or chemically attached scaffolds) and vaccines (natural or modified peptides or nucleic acids, etc.) by techniques known to people familiar with these arts and practices. Interestingly, most of the known CETP antibodies (Roy et al., 1996) target epitopes in the C-terminal half of CETP, further highlight the value of our insights and approaches. Thus, in one embodiment, a newly designed monoclonal antibody (antibody-N) is described against this N-terminal domain which inhibits the CETP interaction to HDL.

Secondly, our studies suggest that the LDL/VLDL binding end generally defined by amino acids 288-319 (GRLMLSLMGDEFKAVLETWGFNTNQEIFQEVVGGFPSQA, SEQ ID NO:5) and 349-360 (FLFPRPDQQHSVA; SEQ ID NO:6) in CETP. By the aforementioned approaches, the CETP transfer activity can also be modified and utilized. These approaches, as well as the ones already known, can obviously be combined, mixed-and-matched, and partially modified to achieve the best therapeutic effects for the substance matters. In one embodiment, a polyclonal antibody, H300, against this C-terminal domain is described which inhibits the CETP interaction to HDL.

While the potential of antibody or vaccine approaches to inhibit CETP and raise HDL cholesterol is known, previous publications and attempts focused on the utilization of the full-length CETP protein or its C-terminal helical region of CETP (˜26 amino acids) as the targeted epitope. Our new studies suggest that the previously described epitope is not the determinant for HDL interaction, which is consistent with the partial inhibition and the enhanced binding of the TP2 antibody that targets the polar side of the C-terminal helix described by Swenson et al., “Mechanism of cholesteryl ester transfer protein inhibition by a neutralizing monoclonal antibody and mapping of the monoclonal antibody epitope,” J Biol Chem. 1989 Aug. 25; 264(24):14318-26). Rather, our findings show that the loops at the N-terminal tip, most significantly Tyr101-Asp110, are primarily responsible for such functions. Since inhibiting the HDL-cholesterol-lowering effect of CETP is expected to be of important therapeutic value, methods and compositions using this previously unrecognized epitope offer a distinctly new mode of action to provide drug candidates to raise HDL levels and treat diseases.

In addition, the new insights on CETP-lipoprotein binding provide methods for eliciting and assaying (e.g. standard assay in the presence of known competitive binders, Surface Plasmon Resonance binding to peptides, direct observations from cryo-EM) candidate agents. Since the functions of CETP and various lipoproteins are very complex, selecting agents with specific binding epitope and correlation their differentiating effects in vitro and in vivo can enable the choice of the most desirable “mode of action” and increase the chance of success in the clinic.

Polyclonal and monoclonal antibodies can be made by well-known methods in the art. Anti-N-terminus CETP or anti-C-terminus CETP antibodies can be made by general methods known in the art and as described in U.S. Pat. Nos. 5,652,340 and 5,869,621, both which are hereby incorporated by reference in their entirety for all purposes. As used herein, the terms “Anti-N-terminus CETP antibody” or “anti-C-terminus CETP antibody” refer to antibodies targeting epitopes described herein as involved in CETP binding and/or interaction with HDL, most notably, epitopes comprising or derived from sequences from human CETP at loops 44-61, 95-116, 151-171, 288-319, 349-360, and possibly beta-strands D42-E46, K56-H60, K94-K98, D114-E115 of CETP. A preferred method of generating these antibodies is by first synthesizing peptide fragments from the N-terminus and/or C-terminus regions of CETP, e.g., 7mer to 15mer peptides from CETP loops 44-61 (ITGEKAMMLLGQVKYGLH; SEQ ID NO:1), 95-116 (GTLKYGYTTAWWLGIDQSIDFE; SEQ ID NO:2), and 151-171 (LLHLQGEREPGWIKQLFTNFI; SEQ ID NO:3) or 288-319 (GRLMLSLMGDEFKAVLETWGFNTNQEIFQEVVGGFPSQA, SEQ ID NO:5) and 349-360(FLFPRPDQQHSVA; SEQ ID NO:6). These peptide fragments should likely cover unique regions in the CETP gene which are involved in CETP lipoprotein binding, such as peptides SEQ ID NO: 4 and SEQ ID NO: 7. If a specific type of modification is found in CETP-lipoprotein binding, a peptide with proper modification can be synthesized. Since synthesized peptides are not always immunogenic by their own, the peptides should be conjugated to a carrier protein before use. Appropriate carrier proteins include but are not limited to Keyhole limpet hemacyanin (KLH). The conjugated phospho peptides should then be mixed with adjuvant and injected into a mammal, preferably a rabbit through intradermal injection, to elicit an immunogenic response. Samples of serum can be collected and tested by ELISA assay to determine the titer of the antibodies and then harvested.

In one embodiment, a specific epitope by an anti-N-terminus CETP or anti-C-terminus CETP antibody can be targeted. A small peptide derived from any of SEQ ID NOS:1-6 can be synthesized having the same amino acid sequence as the targeted epitope region and antibodies specific for this epitope can also be made. For example, in one embodiment, a 7mer to 15-mer peptide peptide derived from loop 95-116 (SEQ ID NO:1) containing at least W105-W106. In another embodiment, a 15-mer peptide, KYGYTTAWWLGIDQS (SEQ ID NO:4), derived from SEQ ID NO:1. In another embodiment, a 10-mer peptide YTTAWWLGID (SEQ ID NO:7), which is CETP Tyr101-Asp110, is synthesized and used for making an antibody. Such antibodies will greatly aid in inhibiting very specific regions of the N-terminal or C-terminal loops identified as involved in CETP-lipoprotein binding to thereby raise HDL levels and treat. Antibodies of the present invention should be able to distinguish N-terminus CETP epitopes from C-terminus CETP epitopes.

Polyclonal (e.g., anti-N-terminus CETP or anti-C-terminus CETP) antibodies can be purified by passing the harvested antibodies through an affinity column Monoclonal antibodies are preferred over polyclonal antibodies and can be generated according to standard methods known in the art of creating an immortal cell line which expresses the antibody. In one embodiment, a CETP antibody as a control is an antibody of U.S. Pat. Nos. 6,410,020 and/or 6,140,474, both of which are hereby incorporated by reference.

Nonhuman antibodies are highly immunogenic in human thus limiting their therapeutic potential. In order to reduce their immunogenicity, nonhuman antibodies need to be humanized for therapeutic application. Through the years, many researchers have developed different strategies to humanize the nonhuman antibodies. One such example is using “HuMAb-Mouse” technology available from MEDAREX, Inc. and disclosed by van de Winkel, in U.S. Pat. No. 6,111,166 and hereby incorporated by reference in its entirety. “HuMAb-Mouse” is a strain of transgenic mice which harbor the entire human immunoglobin (Ig) loci and thus can be used to produce fully human monoclonal antibodies such as monoclonal anti-N-terminus CETP antibodies.

Descriptions of methods and processes for making, testing and using monoclonal antibodies to full-length human CETP are described by Kamada et al, in U.S. Pat. Nos. 6,410,020 and 6,140,474, both of which are hereby incorporated by reference in their entireties for all purposes, which may be applied in the present invention by one having skill in the art. Further descriptions of methods and processes for making, testing and using monoclonal antibodies to specific non-terminal regions of human CETP and methods of modulating CETP activity are described by Rittershaus et al in U.S. Pat. No. 7,078,036, hereby incorporated by reference in its entirety for all purposes, may also be applied in the present invention by one having skill in the art.

The antibody or fragment thereof of the present invention comprises at least one (or 2, 3, 4, 5, or 6) complementarity determining region (CDR) of the V_{H} and/or V_{L} region of an antibody or fragment thereof comprising the amino acid sequence that specifically recognizes the N-terminal or C-terminal regions of CETP. Alternatively, and/or in addition the antibody of the invention comprises at least 1, 2 or 3 CDR(s) of the V_{L} region of an immunoglobulin chain that binds to the N- and/or C-termini of CETP.

A person skilled in the art knows that each variable domain (the heavy chain V_{H} and light chain V_{L}) of an antibody comprises three hypervariable regions, sometimes called complementarity determining regions or “CDRs” flanked by four relatively conserved framework regions or “FRs”. The CDRs contained in the variable regions of the antibody of the invention can be determined, e.g., according to Kabat, Sequences of Proteins of Immunological Interest (U.S. Department of Health and Human Services, third edition, 1983, fourth edition, 1987, fifth edition 1990). The person skilled in the art will readily appreciate that the variable domain of the antibody having the above-described variable domain can be used for the construction of other polypeptides or antibodies of desired specificity and biological function. Thus, the present invention also encompasses polypeptides and antibodies comprising at least one CDR of the above-described variable domain and which advantageously has substantially the same or similar binding properties as the antibody described in the appended examples. The person skilled in the art will readily appreciate that using the variable domains or CDRs described above antibodies can be constructed according to methods known in the art, e.g., as described in EP-A1 0 451 216 and EP-A1 0 549 581.

In some embodiments of the invention, said antibody is a monoclonal antibody, a polyclonal antibody, a single chain antibody, or fragment thereof that specifically binds said N-terminus CETP or C-terminus CETP also including bispecific antibody, synthetic antibody, antibody fragment, such as Fab, Fv or scFv fragments etc., or a chemically modified derivative of any of these. Monoclonal antibodies can be prepared, for example, by the techniques as originally described in Kohler and Milstein, Nature 256 (1975), 495, and Galfre, Meth. Enzymol. 73 (1981), 3, which comprise the fusion of mouse myeloma cells to spleen cells derived from immunized mammals with modifications developed by the art. Furthermore, antibodies or fragments thereof to the aforementioned epitopes can be obtained by using methods which are described, e.g., in Harlow and Lane “Antibodies, A Laboratory Manual”, CSH Press, Cold Spring Harbor, 1988. When derivatives of said antibodies are obtained by the phage display technique, surface plasmon resonance as employed in the BIAcore system can be used to increase the efficiency of phage antibodies which bind to an epitope of the N-terminal or C-terminal regions of CETP (Schier, Human Antibodies Hybridomas 7 (1996), 97-105; Malmborg, J. Immunol. Methods 183 (1995), 7-13). The production of chimeric antibodies is described, for example, in WO89/09622. As discussed above, the antibody of the invention may exist in a variety of forms besides complete antibodies; including, for example, Fv, Fab and F(ab)2, as well as in single chains; see e.g. WO88/09344. In case of bispecific antibodies where one specificity is directed to the N-terminus CETP and the other is directed to the C-terminus of CETP.

The antibodies of the present invention or their corresponding immunoglobulin chain(s) can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to the person skilled in the art; see, e.g., Sambrook, Molecular Cloning A Laboratory Manual, Cold Spring Harbor Laboratory (1989) N.Y.

In another embodiment the present invention relates to a polynucleotide encoding at least a variable region of an immunoglobulin chain of any of the before described antibodies of the invention. One form of immunoglobulin constitutes the basic structural unit of an antibody. This form is a tetramer and consists of two identical pairs of immunoglobulin chains, each pair having one light and one heavy chain. In each pair, the light and heavy chain variable regions or domains are together responsible for binding to an antigen, and the constant regions are responsible for the antibody effector functions. In addition to antibodies, immunoglobulins may exist in a variety of other forms (including less than full-length that retain the desired activities), including, for example, Fv, Fab, and F(ab′)2, as well as single chain antibodies (e.g., Huston, Proc. Nat. Acad. Sci. USA 85 (1988), 5879-5883 and Bird, Science 242(1988), 423-426); see also supra. An immunoglobulin light or heavy chain variable domain consists of a “framework” region interrupted by three hypervariable regions, also called CDR's; see supra.

The antibodies of the present invention can be produced by expressing recombinant DNA segments encoding the heavy and light immunoglobulin chain(s) of the antibody invention either alone or in combination.

The polynucleotide of the invention encoding the above described antibody may be, e.g., DNA, cDNA, RNA or synthetically produced DNA or RNA or a recombinantly produced chimeric nucleic acid molecule comprising any of those polynucleotides either alone or in combination. In some embodiments, the polynucleotide is part of a vector. Such vectors may comprise further genes such as marker genes which allow for the selection of said vector in a suitable host cell and under suitable conditions. In some embodiments, the polynucleotide of the invention is operatively linked to expression control sequences allowing expression in prokaryotic or eukaryotic cells. Expression of said polynucleotide comprises transcription of the polynucleotide into a translatable mRNA. Regulatory elements ensuring expression in eukaryotic cells, such as mammalian cells, are well known to those skilled in the art. They usually comprise regulatory sequences ensuring initiation of transcription and optionally poly-A signals ensuring termination of transcription and stabilization of the transcript. Additional regulatory elements may include transcriptional as well as translational enhancers, and/or naturally-associated or heterologous promoter regions. In this respect, the person skilled in the art will readily appreciate that the polynucleotides encoding at least the variable domain of the light and/or heavy chain may encode the variable domains of both immunoglobulinchains or only one. Likewise, said polynucleotides may be under the control of the same promoter or may be separately controlled for expression. Possible regulatory elements permitting expression in prokaryotic host cells comprise, e.g., the PL, lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Beside elements which are responsible for the initiation of transcription such regulatory elements may also comprise transcription termination signals, such as the SV40-poly-A site or the tk-poly-A site, downstream of the polynucleotide. Furthermore, depending on the expression system used leader sequences capable of directing the polypeptide to a cellular compartment or secreting it into the medium may be added to the coding sequence of the polynucleotide of the invention and are well known in the art. The leader sequence(s) is (are) assembled in appropriate phase with translation, initiation and termination sequences, and a leader sequence capable of directing secretion of translated protein, or a portion thereof, into the periplasmic space or extracellular medium. Optionally, the heterologous sequence can encode a fusion protein including a C- or N-terminal identification peptide imparting desired characteristics, e.g., stabilization or simplified purification of expressed recombinant product. In this context, suitable expression vectors are known in the art such as Okayama-Berg cDNA expression vector pcDV1 (Pharmacia), pCDM8, pRc/CMV, pcDNA1, pcDNA3 (Invitrogen), or pSPORT1 (GIBCO BRL).

In some embodiments, the expression control sequences will be eukaryotic promoter systems in vectors capable of transforming or transfecting eukaryotic host cells, but control sequences for prokaryotic hosts may also be used. Once the vector has been incorporated into the appropriate host, the host is maintained under conditions suitable for high level expression of the nucleotide sequences, and, as desired, the collection and purification of the immunoglobulin light chains, heavy chains, light/heavy chain dimers or intact antibodies, binding fragments or other immunoglobulin forms may follow; see, Beychok, Cells of Immunoglobulin Synthesis, Academic Press, N.Y., (1979); see also, e.g., the appended examples.

As described above, the polynucleotide of the invention can be used alone or as part of a vector to express a peptide of interest in cells, in vitro, or in a cell-free system. The polynucleotides or vectors of the invention are introduced into the cells which in turn produce the antibody. Further, the present invention relates to vectors, particularly plasmids, cosmids, viruses and bacteriophages used conventionally in genetic engineering that comprise a polynucleotide encoding a variable domain of an immunoglobulin chain of an antibody of the invention; optionally in combination with a polynucleotide of the invention that encodes the variable domain of the other immunoglobulin chain of the antibody of the invention. In some embodiments, the vector is an expression vector. Methods which are well known to those skilled in the art can be used to construct recombinant vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1989). An example of a cell-free system is the TNT® SP6 High-Yield Wheat Germ Protein Expression System (cell free protein expression) which is based on an optimized wheat germ extract, is a single-tube, coupled transcription/translation system designed to express proteins (commercially available from Promega Corp., Madison, Wis.).

The peptide of interest can be a peptide of any suitable number of amino acids. In some embodiments, the peptide of interest is equal to or less than about 200 amino acid residues in length. In some embodiments, the peptide of interest is equal to or less than about 100 amino acid residues in length. In some embodiments, the peptide of interest is equal to or more than about 200 amino acid residues in length. In some embodiments, the peptide of interest is equal to or more than about 100 amino acid residues in length.

The nucleic acid constructs of the present invention comprise nucleic acid sequences encoding (a) the antibody of the present invention, or (b) peptide derived from specific loops identified in the N-terminal or C-terminal regions of CETP and optionally a peptide of interest. The nucleic acid of the subject enzymes are operably linked to promoters and optionally control sequences such that the subject enzymes are expressed in a host cell cultured under suitable conditions. The promoters and control sequences are specific for each host cell species. In some embodiments, expression vectors comprise the nucleic acid constructs. Methods for designing and making nucleic acid constructs and expression vectors are well known to those skilled in the art.

Sequences of nucleic acids encoding the subject enzymes are prepared by any suitable method known to those of ordinary skill in the art, including, for example, direct chemical synthesis or cloning. For direct chemical synthesis, formation of a polymer of nucleic acids typically involves sequential addition of 3′-blocked and 5′-blocked nucleotide monomers to the terminal 5′-hydroxyl group of a growing nucleotide chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those of ordinary skill in the art and is described in the pertinent texts and literature (e.g., in Matteuci et al. (1980) Tet. Lett. 521:719; U.S. Pat. Nos. 4,500,707; 5,436,327; and 5,700,637). In addition, the desired sequences may be isolated from natural sources by splitting DNA using appropriate restriction enzymes, separating the fragments using gel electrophoresis, and thereafter, recovering the desired nucleic acid sequence from the gel via techniques known to those of ordinary skill in the art, such as utilization of polymerase chain reactions (PCR; e.g., U.S. Pat. No. 4,683,195).

Each nucleic acid sequence encoding the desired subject enzyme or peptide of interest can be incorporated into an expression vector. Incorporation of the individual nucleic acid sequences may be accomplished through known methods that include, for example, the use of restriction enzymes (such as BamHI, EcoRI, HhaI, Xhol, XmaI, and so forth) to cleave specific sites in the expression vector, e.g., plasmid. The restriction enzyme produces single stranded ends that may be annealed to a nucleic acid sequence having, or synthesized to have, a terminus with a sequence complementary to the ends of the cleaved expression vector. Annealing is performed using an appropriate enzyme, e.g., DNA ligase. As will be appreciated by those of ordinary skill in the art, both the expression vector and the desired nucleic acid sequence are often cleaved with the same restriction enzyme, thereby assuring that the ends of the expression vector and the ends of the nucleic acid sequence are complementary to each other. In addition, DNA linkers may be used to facilitate linking of nucleic acids sequences into an expression vector. The nucleotide sequence encoding the N-terminus region of CETP, C-terminus of CETP, or any sequence derived from SEQ ID NO: 1-7.

A series of individual nucleic acid sequences can also be combined by utilizing methods that are known to those having ordinary skill in the art (e.g., U.S. Pat. No. 4,683,195).

For example, each of the desired nucleic acid sequences can be initially generated in a separate PCR. Thereafter, specific primers are designed such that the ends of the PCR products contain complementary sequences. When the PCR products are mixed, denatured, and reannealed, the strands having the matching sequences at their 3′ ends overlap and can act as primers for each other Extension of this overlap by DNA polymerase produces a molecule in which the original sequences are “spliced” together. In this way, a series of individual nucleic acid sequences may be “spliced” together and subsequently transduced into a host microorganism simultaneously. Thus, expression of each of the plurality of nucleic acid sequences is effected.

Individual nucleic acid sequences, or “spliced” nucleic acid sequences, are then incorporated into an expression vector. The invention is not limited with respect to the process by which the nucleic acid sequence is incorporated into the expression vector. Those of ordinary skill in the art are familiar with the necessary steps for incorporating a nucleic acid sequence into an expression vector. A typical expression vector contains the desired nucleic acid sequence preceded by one or more regulatory regions, along with a ribosome binding site, e.g., a nucleotide sequence that is 3-9 nucleotides in length and located 3-11 nucleotides upstream of the initiation codon in E. coli. See Shine et al. (1975) Nature 254:34 and Steitz, in Biological Regulation and Development: Gene Expression (ed. R. F. Goldberger), vol. 1, p. 349, 1979, Plenum Publishing, N.Y.

Regulatory regions include, for example, those regions that contain a promoter and an operator. A promoter is operably linked to the desired nucleic acid sequence, thereby initiating transcription of the nucleic acid sequence via an RNA polymerase enzyme. An operator is a sequence of nucleic acids adjacent to the promoter, which contains a protein-binding domain where a repressor protein can bind. In the absence of a repressor protein, transcription initiates through the promoter. When present, the repressor protein specific to the protein-binding domain of the operator binds to the operator, thereby inhibiting transcription. In this way, control of transcription is accomplished, based upon the particular regulatory regions used and the presence or absence of the corresponding repressor protein. Examples include lactose promoters (LacI repressor protein changes conformation when contacted with lactose, thereby preventing the Lad repressor protein from binding to the operator) and tryptophan promoters (when complexed with tryptophan, TrpR repressor protein has a conformation that binds the operator; in the absence of tryptophan, the TrpR repressor protein has a conformation that does not bind to the operator). Another example is the tac promoter. (See deBoer et al. (1983) Proc. Natl. Acad. Sci. USA, 80:21-25.) As will be appreciated by those of ordinary skill in the art, these and other expression vectors may be used in the present invention, and the invention is not limited in this respect.

Although any suitable expression vector may be used to incorporate the desired sequences, readily available expression vectors include, without limitation: plasmids, such as pSC101, pBR322, pBBR1MCS-3, pUR, pEX, pMR100, pCR4, pBAD24, pUC19; bacteriophages, such as M13 phage and λ phage. Of course, such expression vectors may only be suitable for particular host cells. One of ordinary skill in the art, however, can readily determine through routine experimentation whether any particular expression vector is suited for any given host cell. For example, the expression vector can be introduced into the host cell, which is then monitored for viability and expression of the sequences contained in the vector. In addition, reference may be made to the relevant texts and literature, which describe expression vectors and their suitability to any particular host cell.

The expression vectors of the invention must be introduced or transferred into the host cell. Such methods for transferring the expression vectors into host cells are well known to those of ordinary skill in the art. For example, one method for transforming E. coli with an expression vector involves a calcium chloride treatment wherein the expression vector is introduced via a calcium precipitate. Other salts, e.g., calcium phosphate, may also be used following a similar procedure. In addition, electroporation (i.e., the application of current to increase the permeability of cells to nucleic acid sequences) may be used to transfect the host microorganism. Also, microinjection of the nucleic acid sequencers) provides the ability to transfect host microorganisms. Other means, such as lipid complexes, liposomes, and dendrimers, may also be employed. Those of ordinary skill in the art can transfect a host cell with a desired sequence using these or other methods.

For identifying a transfected host cell, a variety of methods are available. For example, a culture of potentially transfected host cells may be separated, using a suitable dilution, into individual cells and thereafter individually grown and tested for expression of the desired nucleic acid sequence. In addition, when plasmids are used, an often-used practice involves the selection of cells based upon antimicrobial resistance that has been conferred by genes intentionally contained within the expression vector, such as the amp, gpt, neo, and hyg genes, or curing of an auxotrophy.

The polynucleotides and vectors of the invention can be reconstituted into liposomes for delivery to cells. The vectors containing the polynucleotides of the invention (e.g., the heavy and/or light variable domain(s) of the immunoglobulin chains encoding sequences and expression control sequences) can be transferred into the host cell by well-known methods, which vary depending on the type of cellular host. For example, calcium chloride transfection is commonly utilized for prokaryotic cells, whereas calcium phosphate treatment or electroporation may be used for other cellular hosts; see Sambrook, supra.

The present invention furthermore relates to host cells transformed with a polynucleotide or vector of the invention. The polynucleotide or vector of the invention which is present in the host cell may either be integrated into the genome of the host cell or it may be maintained extrachromosomally. The host cell can be any prokaryotic or eukaryotic cell, such as a bacterial, insect, fungal, plant, animal or human cell. The fungal cells can be of the genus Saccharomyces, in particular those of the species S. cerevisiae. The term “prokaryotic” is meant to include all bacteria which can be transformed or transfected with a DNA or RNA molecules for the expression of an antibody of the invention or the corresponding immunoglobulin chains. Prokaryotic hosts may include gram negative as well as gram positive bacteria such as, for example, E. coli, S. typhimurium, Serratia marcescens and Bacillus subtilis. The term “eukaryotic” is meant to include yeast, higher plant, insect and preferably mammalian cells. Depending upon the host employed in a recombinant production procedure, the antibodies or immunoglobulin chains encoded by the polynucleotide of the present invention may be glycosylated or may be non-glycosylated. Antibodies of the invention or the corresponding immunoglobulin chains may also include an initial methionine amino acid residue. A polynucleotide of the invention can be used to transform or transfect the host using any of the techniques commonly known to those of ordinary skill in the art. Furthermore, methods for preparing fused, operably linked genes and expressing them in, e.g., mammalian cells and bacteria are well-known in the art (Sambrook, Molecular Cloning: A Laboratory Manual, Cold Spring Harbor Laboratory, Cold Spring Harbor, N.Y., 1989). The genetic constructs and methods described therein can be utilized for expression of the antibody of the invention or the corresponding immunoglobulin chains in eukaryotic or prokaryotic hosts. In general, expression vectors containing promoter sequences which facilitate the efficient transcription of the inserted polynucleotide are used in connection with the host. The expression vector typically contains an origin of replication, a promoter, and a terminator, as well as specific genes which are capable of providing phenotypic selection of the transformed cells. Furthermore, transgenic animals, preferably mammals, comprising cells of the invention may be used for the large scale production of the (poly)peptide of the invention.

Thus, in a further embodiment, the present invention relates to a method for the production of an antibody or fragment thereof capable of recognizing the N-terminal or C-terminal regions of CETP comprising (a) culturing the cell of the invention; and (b) isolating said antibody or functional fragment or immunoglobulin chain(s) thereof from the culture,

The transformed hosts can be grown in fermentors and cultured according to techniques known in the art to achieve optimal cell growth. Once expressed, the whole antibodies, their dimers, individual light and heavy chains, or other immunoglobulin forms of the present invention, can be purified according to standard procedures of the art, including ammonium sulfate precipitation, affinity columns, column chromatography, gel electrophoresis and the like; see, Scopes, “Protein Purification”, Springer-Verlag, N.Y. (1982). The antibody or its corresponding immunoglobulin chain(s) of the invention can then be isolated from the growth medium, cellular lysates, or cellular membrane fractions. The isolation and purification of the, e.g., microbially expressed antibodies or immunoglobulin chains of the invention may be by any conventional means such as, for example, preparative chromatographic separations and immunological separations such as those involving the use of monoclonal or polyclonal antibodies directed, e.g., against the constant region of the antibody of the invention. It will be apparent to those skilled in the art that the antibodies of the invention can be further coupled to other moieties for, e.g., drug targeting and imaging applications. Such coupling may be conducted chemically after expression of the antibody or antigen to site of attachment or the coupling product may be engineered into the antibody or antigen of the invention at the DNA level. The DNAs are then expressed in a suitable host system, and the expressed proteins are collected and renatured, if necessary.

The present invention also involves a method for producing cells capable of expressing an antibody of the invention or its corresponding immunoglobulin chain(s) comprising genetically engineering cells with the polynucleotide or with the vector of the invention. The cells obtainable by the method of the invention can be used, for example, to test the interaction of the antibody of the invention with its antigen. Furthermore, the invention relates to an antibody of the invention or fragment thereof encoded by a polynucleotide according to the invention or obtainable by the above-described methods or from cells produced by the method described above. The antibodies of the present invention will typically find use individually in treating substantially any disease susceptible to monoclonal antibody-based therapy. In particular, the immunoglobulins can be used for passive immunization or the removal of HCV or unwanted cells or antigens, such as by complement mediated lysis, all without substantial immune reactions (e.g., anaphylactic shock) associated with many prior antibodies. For an antibody of the invention, typical disease states suitable for treatment include chronic HCV infection.

In some embodiments, the antibodies of the present invention are used to quantify, localize, such as immunolocalize or in situ localize, or isolate a lipoprotein of interest that is linked to the N-terminal or C-terminal regions of CETP. The antibodies of the invention are, for example, suited for use in immunoassays in which they can be utilized in liquid phase or bound to a solid phase carrier. Examples of immunoassays which can utilize the antigen of the invention are competitive and non-competitive immunoassays in either a direct or indirect format. Examples of such immunoassays are the radioimmunoassay (RIA), the sandwich (immunometric assay) and the Western blot assay. The antibodies of the invention can be bound to many different carriers and used to inhibit CETP-lipoprotein binding and interaction. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. The nature of the carrier can be either soluble or insoluble for the purposes of the invention.

There are many different labels and methods of labeling known to those of ordinary skill in the art. Examples of the types of labels which can be used in the present invention include enzymes, radioisotopes, colloidal metals, fluorescent compounds, chemiluminescent compounds, and bioluminescent compounds; see also the embodiments discussed hereinabove.

The present invention also comprises methods of detecting the presence of the N-terminal or C-terminal regions of CETP, or a lipoprotein bound to the N-terminal or C-terminal regions of CETP, in a sample, comprising a sample, contacting said sample with one of the aforementioned antibodies, such as under non-reducing conditions permitting binding of the antibody to the N-terminal or C-terminal regions of CETP, and detecting the presence of the antibody so bound, for example, using immuno assay techniques such as radioimmunoassay or enzymeimmunoassay.

The term “affinity chromatography” in the present invention means chromatography for separation or purification of human CETP contained in a sample by using the affinity between the antigen and antibody. As the examples of a sample, body fluids such as plasma, culture supernatants, or centrifugation supernatants are given. Specifically, the following methods are given as examples.

In one embodiment, a method for separating the human CETP in the sample comprises applying a sample to an insoluble carrier such as a filter or a membrane on which a monoclonal antibody or its fragment of the present invention, which is reactive to the N-terminus or C-terminus of human CETP, has been immobilized to separate the human CETP.

In another embodiment, a method for separating or purifying the human CETP bound to a lipoprotein (e.g., VLDL or HDL) in the sample, comprising immobilizing a monoclonal antibody or its fragment of the present invention which is reactive to the N-terminus or C-terminus of human CETP, to the above-mentioned insoluble carrier (e.g., a cellulose type carrier, an agarose type carrier, a polyacrylamide type carrier, a dextran type carrier, a polystyrene type carrier, a polyvinyl alcohol type carrier, a polyamino acid type carrier and a porous silica type carrier) by known methods (such as physical adsorption, polymerization by cross-linking, trapping in the carrier matrix, or immobilization by non-covalent bonding), filling the insoluble carrier into a column such as a glass, plastic or stainless column having a cylindrical configuration, and applying a sample (e.g., a body fluid such as blood plasma, a culture supernatant, or a centrifugation supernatant) into the column for elution.

In some embodiments of the insoluble carriers for affinity chromatography, any type carrier may be used as long as they can immobilize the monoclonal antibody or its fragment of the present invention on them. As examples, commercially available carriers such as SEPHAROSE 2B, SEPHAROSE 4B, SEPHAROSE 6B, CNBr-SEPHAROSE 4B, AH-SEPHAROSE 4B, CH-SEPHAROSE 4B, ACTIVATED CH-SEPHAROSE 4B, EPDXY-ACTIVATED SEPHAROSE 6B, ACTIVATED THIOL-SEPHAROSE 4B, SEPHADEX, CM-SEPHADEX, ECH-SEPHAROSE 4B, EAH-SEPHAROSE 4B, NHS-ACTIVATED SEPHAROSE, THIOPROPYL SEPHAROSE 6B, and so forth (Pharmacia); BIO-GEL A, CELLEX, CELLEX AE, CELLEX-DM, CELLEX PAB, BIO-GEL-P, HYDRAZIDE BIO-GEL P, AMINOETHYL BIOGEL P, BIO-GEL CM, AFFI-GEL 10, AFFI-GEL 15, AFFI-PREP 10, AFFI-GEL Hz, AFFI-PREP Hz, AFFI-GEL 102, CM BIO-GEL A, AFFI-GEL HEPARIN, AFFI-GEL 501, or AFFI-GEL 601, and so forth (Bio-Rad); CHROMA-GEL A, CHROMA-GEL P, ENZAFIX P-Hz, ENZAFIX P-SH, ENZAFIX P-AB, and so forth (Wako Pure Chemicals); Ae-CELLULOSE, CM-CELLULOSE, PAB CELLULOSE, and so forth (Serva) are given.

In another embodiment, a pharmaceutical composition comprising the monoclonal antibody or its fragment of the present invention as an active ingredient, and may further comprise one or more pharmaceutically acceptable carrier(s) such as excipients, diluents, vehicles, disintegrators, stabilizers, preservatives, buffering agents, emulsifiers, aromatics, coloring agents, sweetening agents, thickening agents, flavoring agents, solubilizing agents, and other additives. Such a pharmaceutical composition may be formed as tablets, pills, powders, granules, injections, liquid preparations, capsules, troches, elixirs, suspensions, emulsions, or syrups. The pharmaceutical composition may be administrated, for example, orally or parentally.

In particular, injections may be prepared by dissolving or suspending the monoclonal antibody or its fragment of the present invention in a pharmaceutically acceptable carrier without toxicity at a concentration from 0.1 μg of the monoclonal antibody/ml of carrier to 10 mg of the antibody/ml of carrier such as physiological saline, and distilled water for injections. Such injections may be administrated to patients who need treatments at dosages of 1 μg to 100 mg/kg of body weight, preferably at 50 μg to 50 mg/kg of body weight from one to several times per day. This administration is performed via clinically suitable routes such as intravenously, subcutaneously, intradermally, intramuscularly, in intraperitoneally and so forth. Preference may be given to intravenous administration.

The pharmaceutical composition of the present invention may be applicable not only for treating or preventing hyperlipidemia but also for treating or preventing various diseases such as arteriosclerosis caused by the abnormal kinetics of CETP, hyperalphalipoproteinemia and hypercholesterolemia.

In one embodiment, two antibodies are administered targeting both the CETP N-terminus and the CETP C-terminus. In some embodiments, the anti-N-terminus CETP and anti-C-terminus CETP antibodies are administered in parallel or in combination with a drug targeting lipoproteins.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Using Electron Microscopy to Determine CETP Regions Involved in Lipoprotein Binding and Interaction

One difficulty in investigating CETP mechanisms using structural methods is that interaction with CETP can alter the size, shape, and composition of lipoproteins, especially HDL (Zhang, L. et al. Morphology and structure of lipoproteins revealed by an optimized negative-staining protocol of electron microscopy. J Lipid Res 52, 175-84 (2011); Chen, B. et al. Apolipoprotein AI tertiary structures determine stability and phospholipid-binding activity of discoidal high-density lipoprotein particles of different sizes. Protein Sci 18, 921-35 (2009); Silva, R. A. et al. Structure of apolipoprotein A-I in spherical high density lipoproteins of different sizes. Proc Natl Acad Sci USA 105, 12176-81 (2008)). We validated an optimized negative-staining electron microscopy (NS-EM) protocol (Zhang, L. et al. An optimized negative-staining protocol of electron microscopy for apoE4 POPC lipoprotein. J Lipid Res 51, 1228-36 (2010)) in which flash-fixation of lipoprotein particles preserves a near native-state conformation for direct visualization of individual molecular or macromolecular particles. We applied this protocol to study the mechanisms by which CETP interacts with human plasma HDL, LDL and VLDL. Three-dimensional (3D) reconstructions of CETP, free and HDL-bound, were obtained by single-particle techniques. In addition, we used inhibitory CETP antibodies to identify the regions of CETP that interact with HDL, LDL, and VLDL. Finally molecular dynamics (MD) simulation was used to assess the molecular mobility of CETP and predict the likely conformational changes that are associated with lipid transfer.

Conventional cryo-electron microscopy (cryo-EM) is often the method of choice for studies of protein structure under physiological conditions because it avoids the artifact of rouleaux formation that are induced by fixatives and stains (Zhang, L. et al. J Lipid Res 51, 1228-36 (2010)). Still, cryo-EM studies of CETP are challenging; small molecules (<200 kDa) are difficult to image or reconstruct by the cryo-EM single-particle approach because of low-contrast (Ohi, M., Li, Y., Cheng, Y. & Walz, T. Negative Staining and Image Classification—Powerful Tools in Modern Electron Microscopy. Biol Proced Online 6, 23-34 (2004)). Thus, we studied human recombinant CETP by using optimized negative-staining (NS) and a cryo-positive-staining (cryo-PS) EM protocol.

Our optimized NS protocol, refined from the conventional NS protocol, which eliminates rouleaux-artifact of lipoprotein particles, was statistically validated as a way to determine lipoprotein particle shapes and sizes (Zhang, L. et al. Morphology and structure of lipoproteins revealed by an optimized negative-staining protocol of electron microscopy. J Lipid Res 52, 175-84 (2011); Zhang, L. et al. An optimized negative-staining protocol of electron microscopy for apoE4 POPC lipoprotein. J Lipid Res 51, 1228-36 (2010)). The cryo-PS-EM was modified from Adrian's cryo-negative-stain (cryo-NS) protocol (described in Adrian, M., Dubochet, J., Fuller, S. D. & Harris, J. R. Cryo-negative staining. Micron 29, 145-60 (1998)) by combining our optimized NS and conventional cryo-EM protocols. The cryo-EM protocols were described in Ren, G., Reddy, V. S., Cheng, A., Melnyk, P. & Mitra, A. K. Visualization of a water-selective pore by electron crystallography in vitreous ice. Proc Natl Acad Sci USA 98, 1398-403 (2001); Ren, G., Cheng, A., Reddy, V., Melnyk, P. & Mitra, A. K. Three-dimensional fold of the human AQP1 water channel determined at 4 A resolution by electron crystallography of two-dimensional crystals embedded in ice. J Mol Biol 301, 369-87 (2000); and Ren, G. et al. Model of human low-density lipoprotein and bound receptor based on cryoEM. Proc Natl Acad Sci USA 107, 1059-64 (2010), hereby incorporated by reference. Instead of air-drying the sample in the last step of the NS protocol, the sample was flash-frozen in liquid nitrogen temperature. Since the cryo-EM image of particle has reversed contrast to that from the Adrian's cryo-NS protocol, but has consistent contrast to that from conventional cryo-EM image, we call this the cryo-PS-EM protocol.

We compared the particle shape and size of images of CETP with the CETP crystal structure [PDB entry 2OBD in Qiu, X. et al. Crystal structure of cholesteryl ester transfer protein reveals a long tunnel and four bound lipid molecules. Nat Struct Mol Biol 14, 106-13 (2007)]. Survey optimized NS-EM micrographs and selected particle views reveal the expected banana-shaped CETP with dimensions of ˜125×30 Å (data not shown). When the CETP crystal structure is overlaid onto a reference-free class average of NS-EM images, a near perfect match in shape and size is found (data not shown), and interestingly, even the concave surface, C-terminal end (more globular) and N-terminal end (more tapered) of CETP are readily distinguished (data not shown). These studies validate direct NS-EM as a way to visualize the structure of CETP in other settings where it associates with various lipoproteins.

Survey cryo-PS-EM micrographs and selected particle views (data not shown) also display the banana-shaped CETP with a shape and dimensions similar to those observed from the optimized NS-EM protocol described herein (data not shown).

Protein Isolation and Purification.

Recombinant human CETP (˜53 kDa before post-translational modifications) from the Qiu laboratory was expressed and purified from the dihydrofolate reductase-deficient Chinese hamster ovary cell line DG44 (Qiu, X. et al. Crystal structure of cholesteryl ester transfer protein reveals a long tunnel and four bound lipid molecules. Nat Struct Mol Biol 14, 106-13 (2007)). The CETP concentration was determined by absorbance assay (280 nm). Discoidal reconstituted HDL (rHDL) consisting of apoA-I purified from pooled samples of normal human plasma, 1-palmitoyl-2-oleoyl phosphatidylcholine (POPC), and unesterified cholesterol (UC) (initial POPC:UC:apoA-I molar ratio was 100:10:1) were prepared by the cholate dialysis method (Cavigiolio, G. et al. The interplay between size, morphology, stability, and functionality of high-density lipoprotein subclasses. Biochemistry 47, 4770-9 (2008)). Discoidal HDL particles were converted into spherical rHDL by incubation with fatty acid-free bovine serum albumin, β-mercaptoethanol, ultracentrifugally isolated LDL and purified lecithin:cholesterol acyltransferase (LCAT) (Rye, K. A. & Barter, P. J. The influence of apolipoproteins on the structure and function of spheroidal, reconstituted high density lipoproteins. J Biol Chem 269, 10298-303 (1994)). The spherical HDL species were isolated in the Rye laboratory by sequential ultracentrifugation in the 1.07<d<1.21 g/ml density range as previously described (Rye, K. A. & Barter, P. J. The influence of apolipoproteins on the structure and function of spheroidal, reconstituted high density lipoproteins. J Biol Chem 269, 10298-303 (1994)). The apoA-I concentration in the spherical HDL preparations was determined on a Hitachi 902 automatic analyzer (Roche Diagnostics, GmbH, Mannheim, Germany) by the bicinchoninic assay using BSA as a standard (Smith, P. K. et al. Measurement of protein using bicinchoninic acid. Anal Biochem 150, 76-85 (1985). LDL (d=1.006-1.069 g/ml, and VLDL (d<1.006 g/ml) were isolated in the Pownall and Krauss laboratories respectively by sequential flotation of plasma from a fasted, healthy male volunteer, and further purified by isopycnic density gradient ultracentrifugation as previously described (Gaubatz, J. W. et al. Dynamics of dense electronegative low density lipoproteins and their preferential association with lipoprotein phospholipase A(2). J Lipid Res 48, 348-57 (2007)). The protein concentrations of the LDL and VLDL were determined by absorbance assay (280 nm). The polycolonial CETP antibodies, H300 and N13, were purchased from Santa Cruz Biotechnology, Inc., CA.

Binary Complex Formation.

To prepare the CETP•HDL complexes, CETP (final concentration 0.93 mg/ml, i.e. 17.5 μM) and HDL (final apoA-I concentration from 2.96 mg/ml, i.e. 35 μM, to 0.30 mg/ml, i.e. 3.5 μM) were incubated at 37° C. for 2D NS-EM, or at 4° C. for 3D reconstructions for 1 hour at molar ratios ranging from 0.5:1 to 5:1 (CETP:HDL, assuming three apoA-I molecules/HDL particle). CETP•LDL and CETP•VLDL complexes were formed similarly using CETP (final concentration 0.6 mg/ml for LDL and 0.23 mg/ml for VLDL), LDL (final apoB-100 concentration 3.1 mg/ml, i.e. 5.6 μM), and VLDL (final protein concentration 1.3 mg/ml, i.e. 2.1 μM) at molar ratios of 2:1 (CETP:LDL) and 2:1 (CETP:VLDL), respectively. Although the apolipoprotein content of VLDL varies, VLDL contains one apo B-100 molecule, which is ˜550 kDa and significantly larger than other the apolipoproteins in these particles (E: 35 kDa, A-I: 28 kDa, C-I, II, III: <10 kDa). Thus, a reasonable estimation of the molecular mass of proteins contained in VLDL is ˜600 kDa for the calculation of VLDL molarities. All samples were examined and imaged with either a FEI Tecnai T20 (Philips Electron Optics/FEI, Eindhoven, The Netherlands) or a Zeiss Libra 120 transmission electron microscope (Carl Zeiss NTS GmbH, Germany).

Ternary Complex Formation.

CETP (final concentration 0.33 mg/ml, i.e. 6.2 μM) was incubated for 30 minutes at 37° C. or 4° C., as described above for binary complexes, with HDL (final apoA-I concentration 0.26 mg/ml, i.e. 3.0 μM) at a molar ratio of 2:1 (CETP:HDL), and LDL (final apoB-100 concentration 0.86 mg/ml, i.e. 1.5 μM) was added at a molar ratio of 2:1 (HDL:LDL). HDL•CETP•VLDL complexes were prepared with VLDL (final protein concentration 0.93 mg/ml, i.e. 1.5 μM) and the HDL•CETP complex at a molar ratio of 2:1 (HDL:VLDL).

Negative-Staining (NS) EM Specimen Preparation by the Optimized NS Protocol.

Specimens were prepared for EM by the optimized NS protocol as described^{14,17}. In brief, CETP (final concentration 0.93 mg/ml, i.e., 17.5 uM) and HDL•CETP (final concentration 0.93 mg/ml) complexes were diluted to 0.005 mg/ml with DPBS buffer. An aliquot (˜3 μl) was placed on a thin-carbon-coated 300 mesh copper grid (Cu-300CN, Pacific Grid-Tech, San Francisco, Calif.) that had been glow-discharged. After ˜1 min, excess solution was blotted with filter paper. The grid was washed by briefly touching the surface of the grid with a drop (˜30 μl) of distilled water on parafilm and blotted dry with filter paper. This touching and blotting step was performed three times, each time with a clean drop of distilled water. Three drops of uranyl formate (UF) negative stain (1%, w/v) on parafilm were then applied successively, and excess stain was removed in the same fashion by blotting. The grid was allowed to remain in contact with the last drop of stain with the sample side down for 1-3 min in the dark before removal of excess stain and was air-dried at room temperature^{14,17}. Since UF solutions are light sensitive and unstable, the newly prepared solution was aliquoted and stored in the dark at −80° C. Just before using, each aliquot was thawed in a water bath in the dark, and then filtered (0.02 μm filter). The filter syringe was wrapped with aluminum foil to protect the UF solution from light. The same protocol was used to prepare other binary and ternary complexes. 1% UF solution was diluted in-house from the UF powder purchased from Structure Probe, Inc. West Chester, Pa.

The lipoprotein particle sizes and shapes obtained from this optimized NS protocol have statistical analysis less than 5% differ from that obtained from in a frozen-hydrated native state by cryo-EM of apoE4 HDL particles (Zhang, L. et al. Morphology and structure of lipoproteins revealed by an optimized negative-staining protocol of electron microscopy. J Lipid Res 52, 175-84 (2011); Zhang, L. et al. An optimized negative-staining protocol of electron microscopy for apoE4 POPC lipoprotein. J Lipid Res 51, 1228-36 (2010)). In compared to the conventional NS protocol¹⁷ that predominately used to exam lipoprotein particles, but result in stain-induced structural artifacts, including the generation of rouleaux, this optimized NS protocol can reduce the rouleaux artifact by using UF instead of phosphotungstic acid (PTA) under a low salt condition.

Assessment of CETP Function by EM.

HDL/CETP/LDL mixtures were prepared by combining CETP (final concentration 0.33 mg/ml), HDL (final apoA-I concentration 0.26 mg/ml), and LDL (final apoB-100 concentration 0.86 mg/ml) at a molar ratio of 4:2:1 (CETP:HDL:LDL) on ice, then incubating them at 37° C. for up to 48 hours in a thermo-incubator. Each aliquot was diluted to an apoA-I concentration of 5 μg/ml with Dulbecco's phosphate-buffered saline (DPBS: 2.7 mM KCl, 1.46 mM KH₂PO₄, 136.9 mM NaCl, and 8.1 mM Na₂HPO₄; Invitrogen), and prepared as negative-staining EM specimens with 1% uranyl formate^{14,17}. Samples of HDL/CETP, LDL/CETP, HDL/LDL, HDL, LDL, H300/HDL/CETP/LDL, and N13/HDL/CETP/LDL were prepared similarly, with a molar ratio of antibody (H300/N13) to CETP of 2:1.

Labeling Lipoprotein•CETP Complexes with Antibodies.

HDL (final apoA-I concentration 0.26 mg/ml) and LDL (final apoB-100 concentration 0.86 mg/ml) were incubated at 4° C. for 0.05-4 hours with two molar equivalents of CETP (final concentration 0.33 mg/ml) and then at 4° C. overnight with the anti-CETP antibodies N13 or H300 (final concentration 1.86 mg/ml) at a molar ratio of 1:2 (CETP:antibody). The sample was diluted so that the CETP concentration was 2 μg/ml and prepared for negative-staining EM specimens within 5 minutes.

3D Reconstruction of the HDL•CETP Complex.

Images were processed with SPIDER, EMAN, and FREALIGN software packages⁴⁰⁻⁴². The defocus and stigmatism of each micrograph were determined by fitting the contrast transfer function (CTF) parameters with its power spectrum by ctffind3 in the FREALIGN software package⁴⁰. Micrographs with poor correlation of phase residuals, large stigmatism (>0.1 μm), or distinguishable drift were excluded. The phase of each micrograph was corrected by a Wiener filter with the SPIDER software package⁴¹. Only isolated particles from the NS-EM images were initially selected and windowed as 256×256 pixel images (˜360×360 Å at the specimen) using the boxer program in the EMAN⁴². We used the same program as that used for further selecting lipoprotein particles with a homogeneous size for 3D reconstruction and refinement that we term “computational size-exclusion gel-filtration” algorithms (Ren, G. et al. Model of human low-density lipoprotein and bound receptor based on cryoEM. Proc Natl Acad Sci USA 107, 1059-64 (2010)). Using this method, ˜38% of the particles in total were used for 3D reconstruction, from which ˜317 class averages were generated by reference-free class averages computed using refind2d.py in EMAN⁴². To prevent bias from a starting model, a featureless, smooth, solid cylinder (length ˜75 Å, diameter ˜35 Å) perpendicularly attached to a featureless, solid Gaussian globule (diameter 120×100×80 Å) was used as an initial starting model. This model was generated based on typical features in reference class averages⁴³. For the first four rounds of refinement, only very low resolution particle information was used (below the first CTF zero in reciprocal space). Iterative refinement proceeded to convergence. Then, CTF amplitude and phase corrections, finer angular sampling, and solvent flattening via masking were performed for higher-resolution refinement. This process was iterated to convergence. According to the 0.5 Fourier shell correlation criterion (Bottcher, B., Wynne, S. A. & Crowther, R. A. Determination of the fold of the core protein of hepatitis B virus by electron cryomicroscopy. Nature 386, 88-91 (1997).), the final resolution of the asymmetric reconstruction of HDL•CETP complex was 14 Å (data not shown).

3D Reconstruction of CETP.

To avoid bias in the initial model for CETP reconstruction, we used a featureless solid cylinder as an initial starting model. The cylinder was 125 Å long and 30 Å in diameter, typical of the structural features displayed in the reference-free class averages. The 3D reconstruction was constructed from 8,879 windowed particles from cryo-PS EM images after CTF correction and by following a protocol similar to that of HDL•CETP complex reconstruction for iteration and convergence. According to the 0.5 Fourier shell correlation criterion, the final resolution of the asymmetric reconstruction for CETP was 13 Å (data not shown).

Statistical Analysis.

For statistical analyses of the HDL•CETP•LDL ternary complexes, all LDL particles in each micrograph were windowed and identified using boxer in the EMAN software package. A total of 5 micrographs were used. Before particle selection, the contrast transfer function (CTF) of each micrograph was determined and then phase corrected by the phase-flip option in ctfit (EMAN software). Ternary complexes were first Gaussian low-pass filtered before being identified and selected by examining the particles at 4× zoom. The criteria to distinguish ternary complexes were as follows: 1) the shortest distance between the surfaces of the LDL and HDL particles should be shorter than longest dimension of CETP, i.e. <130 Å; and 2) there should be an identifiable small connecting density between the HDL and LDL particles. 523 LDL particles were selected from 5 micrographs, in which 130 (˜25%) particles satisfied these criteria. A HDL particle may be randomly distributed around the LDL particle it is bridged to, resulting in the connecting portion being obscured from view. Thus it is reasonable to believe that the actual percentage of LDL particles belonging to a ternary complex is higher than the observed ˜25%. For the statistical analysis of the HDL•CETP•VLDL ternary complexes, a percentage of ˜30% for VLDL particles belonging to a ternary complex was obtained by following a protocol similar to that of HDL•CETP•LDL complexes.

For statistical analysis of particle size in the functional assays, a total of ˜500 HDL and/or LDL particles from each of the various incubation protocols were manually selected from CTF-corrected micrographs as described above. Particle size was determined by measuring diameters along two orthogonal directions, one of which was the longest dimension. The geometric averages of two diameters were calculated and expressed as mean±standard deviation (SD). The Python program was used for data analysis. The absorbance histograms for the pixels in each image were scaled to the mean, and the SD was used as error. To get a clear comparison between various incubation conditions, mean size and SD values were divided by the initial mean size, i.e., the mean size at 3 minutes.

Negative-Staining EM Specimen Preparation.

Specimens were prepared for EM as described⁴ with modifications⁵. CETP (final concentration 0.93 mg/ml) and HDL•CETP complexes were diluted to 0.005 mg/ml with DPBS buffer. Aliquots (˜3 μl) were applied to the 400 mesh glow-discharged thin carbon-coated EM grids (Cu-400CN, Pacific Grid-Tech, USA) as previously described⁵. The same protocol was used to prepare other binary and ternary complexes.

### Example 2

### Antibodies Made to CETP N-Terminal and C-Terminal Regions Inhibit CETP-Lipoprotein Binding and Interaction

CETP N- and C-Terminal Domains Interact with HDL and LDL/VLDL Respectively.

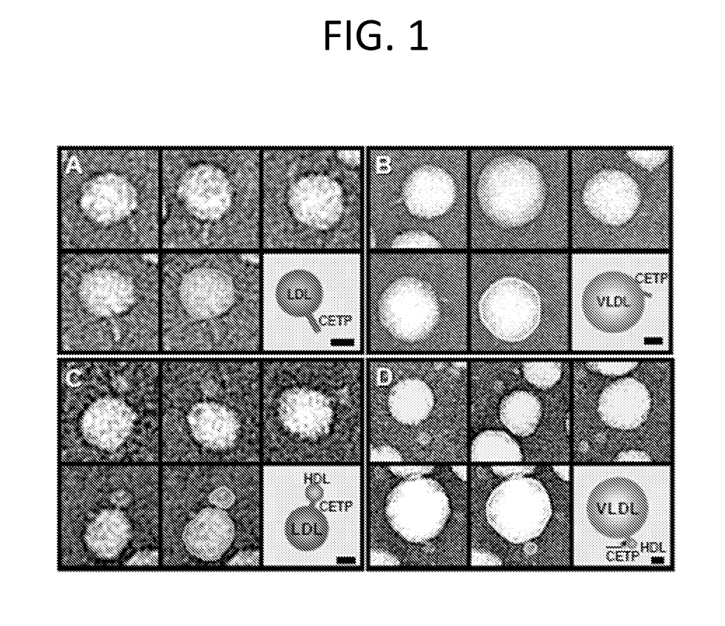

CETP was incubated separately with LDL and VLDL and examined by the optimized NS-EM protocol. Spherical LDL (diameter ˜200-270 Å) and VLDL (diameter ˜370-570 Å) particles were observed with a single CETP protruding from the surfaces as part of a binary complex (FIGS. 1A and B). Although we did not observe LDL•CETP complexes with two protruding CETP molecules, nor two LDLs or VLDLs bridged by one CETP, we did observe occasional VLDL particles with two attached CETP molecules. This is likely due to different surface properties induced by differing apolipoproteins compositions and lipid surface curvatures. Measurements of >100 of these binary complexes revealed that the free-end width of CETP is ˜30 Å, similar to that observed for the HDL•CETP complexes. The free-end lengths on LDL and VLDL are ˜100±10 and ˜105±10 Å respectively, shorter than the length of CETP alone (˜125 Å), suggesting that the hidden portions (˜25±10 and ˜20±10 Å) of CETP penetrate the LDL or VLDL surface respectively (FIGS. 1A and B).

A domain-specific polyclonal CETP antibody, H300 (Liu, J., Bartesaghi, A., Borgnia, M. J., Sapiro, G. & Subramaniam, S. Molecular architecture of native HIV-1 gp120 trimers. Nature 455, 109-13 (2008); Flemming, D., Thierbach, K., Stelter, P., Böttcher, B. & Hurt, E. Precise mapping of subunits in multiprotein complexes by a versatile electron microscopy label. Nature Structural and Molecular Biology 17, 775-778 (2010).), for which the epitopes are within a region containing the entire C-terminal β-barrel domain and part of the central β-sheet (amino acids 194-493), was used to identify the parts of CETP that penetrates lipoproteins. The optimized NS-EM micrographs of the HDL•CETP complex show a characteristic “Y”-shaped density usually near the free-end of CETP. In contrast, LDL•CETP•H300 complexes were rarely seen in the micrographs obtained from the incubations of antibody H300 with LDL and CETP, suggesting that the H300 epitopes are buried within the LDL particle. Moreover, the percentage of binary complexes (LDL•CETP) was also lower than that without H300 (<15% vs. ˜38%), suggesting that H300 inhibits CETP-LDL interaction. These experiments are consistent with the N-terminal domain of CETP interacting with HDL, while the C-terminal domain preferentially interacts with LDL. This is also consistent with the fitting of the crystal structure with the 3D density map and 2D images of the HDL•CETP complex (above section 2), and the dimensions calculated from the CETP pores to the HDL surface (above section 3).

a CETP Bridge Mediates Ternary Complexes of HDL with LDL and VLDL.

After coincubation of CETP, HDL, and LDL, the optimized NS-EM microgrpahs showed ˜25% of LDL particles connecting to HDL particles by a ˜25-55 Å long CETP bridge (FIG. 1C). The length of the bridge is significantly shorter than the length of CETP alone (˜125 Å) indicating that CETP penetrates into one or both lipoprotein surfaces or cores to form the ternary complex. When repeated with VLDL, ˜30% of the VLDL particles were connected to HDL particles by CETP bridges (length ˜35-65 Å, FIG. 1D). Unlike LDL, ˜30% of the VLDL complexes were bound to more than one HDL•CETP complex, likely due to their greater surface area, which provides more “binding sites”; bridges between lipoproteins of the same class were not observed, further supporting the hypothesis that HDL and LDL/VLDL bind to different CETP domains. The coexistence of ternary complexes of HDL•CETP•LDL and HDL•CETP•VLDL and lipid transfer is consistent with the mechanistic model of CE transfer through a tunnel within CETP. These observations do not totally exclude the shuttle mechanism, since single CETP molecules coexisted with HDL and LDL under the conditions of excess CETP that was used. However, the tunnel mechanism seems more plausible because it is supported by the observation of distinct binding sites for HDL and LDL/VLDL on CETP.

CETP Reaction Mechanism.

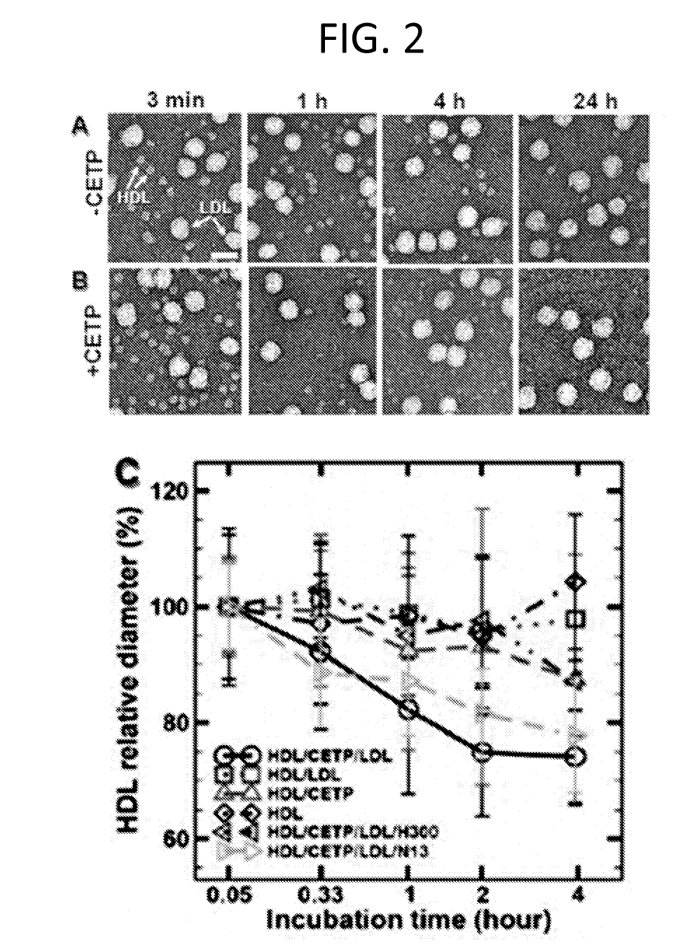

CETP with HDL and/or LDL were incubated at physiological temperatures for up to 48 hours during which HDL size was measured using the optimized NS-EM. When only two of three components (CETP, HDL and LDL) were incubated for up to 4 hours, HDL size did not change (FIGS. 2A and C). However, when all three components were co-incubated, ternary complexes were observed at all incubation times (FIG. 2B), during which the mean HDL particle size decreased by 25.8±8.0% after 4 hours (black circles in FIG. 2C, Table 1). This decrease suggests that the HDL particles are depleted of core lipids by CETP (i.e., there is a net mass transfer of CEs to LDL). The rate constant for CE transfer was 0.58±0.19 h⁻¹ (r²>0.94) based on HDL size changes and the assumption that the decrease in HDL size resulted only from CE outflow. In contrast, the size of the LDL particles did not change noticeably, most likely because the amount of accreted CE is small relative to the LDL particle volume, which is ˜30 times greater than that of HDL. These data suggest that CETP transfers core lipids from HDL to LDL and possibly VLDL, as ternary complexes. These experiments do not support the shuttle mechanism in which CETP dissociates from the HDL surface after it has removed the maximum amount of CE from the HDL core, i.e., after 3-4 hours of incubation. Specifically, with the shuttle mechanism, the percentage of CETP•HDL complexes should decrease over time after incubation of CETP with HDL, something that was not observed even after 48 hours.

We performed additional experiments using CETP polyclonal antibodies H300 and N13. The epitopes for H300 are near the C-terminal end of CETP while those for N13 are near the N-terminal β-barrel domain close to the central β-sheet. The antibodies were incubated separately with HDL, CETP and LDL at 37° C., and aliquots were collected at various times and examined by the optimized NS-EM method. Co-incubation of H300 with LDL and HDL inhibited the decrease in the size of HDL (FIG. 2C and Table 1), from which we conclude that H300 inhibits CE transfer. In contrast, a similar incubation in which H300 was replaced by N13 reduced HDL size ˜18.4% after 4 hours, a value similar to that observed without antibodies (FIG. 2C and Table 1).

EM images utilizing polyclonal antibodies, which recognize multiple epitopes, can be misleading because there could be as many complexes as epitopes. However, measurements of HDL size change are less ambiguous because they reveal the predominant class of epitopes blocked by the array of antibodies. Thus, H300 binds near the CETP C-terminus and in so doing blocks formation of the ternary (HDL•CETP•LDL) and binary (LDL•CETP) complexes and inhibits HDL to LDL CE transfer. Given that our data implicates the N- and C-terminal regions of CETP with lipoproteins, the absence of inhibition by the N-13 antibody, which binds nearer to the central region of CETP on the N terminal side of CETP, was not expected to be inhibitory, as observed.

Although the above results do not favor the shuttle mechanism for CETP-mediated transfer of neutral lipids between HDL and LDL, they do not exclude possibility that CETP shuttles neutral lipids between HDL particles themselves. Although the mean HDL particle size did not change after incubation with CETP for up to 4 hours, the standard deviation at 4 hours was larger than that at 3 minutes (Table 1). The micrographs and histograms show small amounts (<5%) of larger HDL particles (>200 Å), while the remaining HDL particles are smaller in size (data not shown), raising the possibility the CETP may shuttle CE among HDL particles.

### Example 3

### Prevention of Arteriosclerosis by Anti-N-Terminus or Anti-C-Terminus CETP Monoclonal Antibody

Two kinds of purified anti-human N-terminus CETP or anti-human C-terminus CETP monoclonal antibodies, are prepared and dissolved in distilled water for injections at a concentration ratio of for example, 29:1, to prepare injections.

The mixed injectable solution is administrated intraperitoreally to a subject at a dose of for example, 75 mg/kg per injection per day for several days.

The time just before the antibody administration is set as 0. Opthalmo-blood is sampled at days 2, 4, 8, and 11 and the plasma is separated by centrifugation. The amounts of HDL cholesterol in the plasma obtained are determined using standard laboratory lipoprotein panel determination methods.

HDL cholesterol level in blood should rise significantly when the anti-human N-terminus CETP or anti-human C-terminus CETP monoclonal antibody of the present invention is administered in vivo. HDL is considered to be an important lipoprotein having anti-arteriosclerosis effect. Administration of the antibody should prevent or reduce the development of atherosclerosis lesions by the increase of HDL in blood.

### REFERENCES

1. Barter, P. J. et al. Cholesteryl ester transfer protein: a novel target for raising HDL and inhibiting atherosclerosis. Arterioscler Thromb Vasc Biol 23, 160-7 (2003).
2. Camejo, G., Waich, S., Quintero, G., Berrizbeitia, M. L. & Lalaguna, F. The affinity of low density lipoproteins for an arterial macromolecular complex. A study in ischemic heart disease and controls. Atherosclerosis 24, 341-54 (1976).
3. Gordon, T., Castelli, W. P., Hjortland, M. C., Kannel, W. B. & Dawber, T. R. High density lipoprotein as a protective factor against coronary heart disease. The Framingham Study. Am J Med 62, 707-14 (1977).
4. Hayek, T. et al. Hypertriglyceridemia and cholesteryl ester transfer protein interact to dramatically alter high density lipoprotein levels, particle sizes, and metabolism. Studies in transgenic mice. J Clin Invest 92, 1143-52 (1993).
5. Brown, M. L. et al. Molecular basis of lipid transfer protein deficiency in a family with increased high-density lipoproteins. Nature 342, 448-51 (1989).
6. Inazu, A. et al. Increased high-density lipoprotein levels caused by a common cholesteryl-ester transfer protein gene mutation. N Engl J Med 323, 1234-8 (1990).
7. Niesor, E. J. Different effects of compounds decreasing cholesteryl ester transfer protein activity on lipoprotein metabolism. Curr Opin Lipidol (2011).
8. Miyares, M. A. Anacetrapib and dalcetrapib: two novel cholesteryl ester transfer protein inhibitors. Ann Pharmacother 45, 84-94 (2011).
9. Kappelle, P. J., van Tol, A., Wolffenbuttel, B. H. & Dullaart, R. P. Cholesteryl Ester Transfer Protein Inhibition in Cardiovascular Risk Management: Ongoing Trials will End the Confusion. Cardiovasc Ther (2011).
10. Qiu, X. et al. Crystal structure of cholesteryl ester transfer protein reveals a long tunnel and four bound lipid molecules. Nat Struct Mol Biol 14, 106-13 (2007).
11. Barter, P. J. & Jones, M. E. Kinetic studies of the transfer of esterified cholesterol between human plasma low and high density lipoproteins. J Lipid Res 21, 238-49 (1980).
12. Ihm, J., Quinn, D. M., Busch, S. J., Chataing, B. & Harmony, J. A. Kinetics of plasma protein-catalyzed exchange of phosphatidylcholine and cholesteryl ester between plasma lipoproteins. J Lipid Res 23, 1328-41 (1982).
13. Tall, A. R. Plasma cholesteryl ester transfer protein. J Lipid Res 34, 1255-74 (1993).
14. Zhang, L. et al. Morphology and structure of lipoproteins revealed by an optimized negative-staining protocol of electron microscopy. J Lipid Res 52, 175-84 (2011).
15. Chen, B. et al. Apolipoprotein AI tertiary structures determine stability and phospholipid-binding activity of discoidal high-density lipoprotein particles of different sizes. Protein Sci 18, 921-35 (2009).
16. Silva, R. A. et al. Structure of apolipoprotein A-I in spherical high density lipoproteins of different sizes. Proc Natl Acad Sci USA 105, 12176-81 (2008).
17. Zhang, L. et al. An optimized negative-staining protocol of electron microscopy for apoE4 POPC lipoprotein. J Lipid Res 51, 1228-36 (2010).
18. Ohi, M., Li, Y., Cheng, Y. & Walz, T. Negative Staining and Image Classification—Powerful Tools in Modern Electron Microscopy. Biol Proced Online 6, 23-34 (2004).
19. Adrian, M., Dubochet, J., Fuller, S. D. & Harris, J. R. Cryo-negative staining. Micron 29, 145-60 (1998).
20. Ren, G., Reddy, V. S., Cheng, A., Melnyk, P. & Mitra, A. K. Visualization of a water-selective pore by electron crystallography in vitreous ice. Proc Natl Acad Sci USA 98, 1398-403 (2001).
21. Ren, G., Cheng, A., Reddy, V., Melnyk, P. & Mitra, A. K. Three-dimensional fold of the human AQP1 water channel determined at 4 A resolution by electron crystallography of two-dimensional crystals embedded in ice. J Mol Biol 301, 369-87 (2000).
22. Ren, G. et al. Model of human low-density lipoprotein and bound receptor based on cryoEM. Proc Natl Acad Sci USA 107, 1059-64 (2010).
23. Hayat, M. A. & Miller, S. E. Negative Staining, (Mcgraw-Hill (Tx), 1990).
24. Morton, R. E. & Greene, D. J. The surface cholesteryl ester content of donor and acceptor particles regulates CETP: a liposome-based approach to assess the substrate properties of lipoproteins. J Lipid Res 44, 1364-72 (2003).
25. Swenson, T. L., Brocia, R. W. & Tall, A. R. Plasma cholesteryl ester transfer protein has binding sites for neutral lipids and phospholipids. J Biol Chem 263, 5150-7 (1988).
26. Pettersen, E. F. et al. UCSF Chimera—a visualization system for exploratory research and analysis. J Comput Chem 25, 1605-12 (2004).
27. Liu, J., Bartesaghi, A., Borgnia, M. J., Sapiro, G. & Subramaniam, S. Molecular architecture of native HIV-1 gp120 trimers. Nature 455, 109-13 (2008).
28. Flemming, D., Thierbach, K., Stelter, P., Böttcher, B. & Hurt, E. Precise mapping of subunits in multiprotein complexes by a versatile electron microscopy label. Nature Structural and Molecular Biology 17, 775-778 (2010).
29. Phillips, J. C. et al. Scalable molecular dynamics with NAMD. J Comput Chem 26, 1781-802 (2005).
30. Atilgan, A. R. et al. Anisotropy of fluctuation dynamics of proteins with an elastic network model. Biophys J 80, 505-15 (2001).
31. Doruker, P., Atilgan, A. R. & Bahar, I. Dynamics of proteins predicted by molecular dynamics simulations and analytical approaches: application to alpha-amylase inhibitor. Proteins 40, 512-24 (2000).
32. Weers, P. M. & Ryan, R. O. Apolipophorin III: role model apolipoprotein. Insect Biochem Mol Biol 36, 231-40 (2006).
33. Saito, H. et al. Domain structure and lipid interaction in human apolipoproteins A-I and E, a general model. J Biol Chem 278, 23227-32 (2003).
34. Han, M. et al. Disruption of human plasma high-density lipoproteins by streptococcal serum opacity factor requires labile apolipoprotein A-I. Biochemistry 48, 1481-7 (2009).
35. Wang, S., Kussie, P., Deng, L. & Tall, A. Defective binding of neutral lipids by a carboxyl-terminal deletion mutant of cholesteryl ester transfer protein. Evidence for a carboxyl-terminal cholesteryl ester binding site essential for neutral lipid transfer activity. J Biol Chem 270, 612-8 (1995).
36. Cavigiolio, G. et al. The interplay between size, morphology, stability, and functionality of high-density lipoprotein subclasses. Biochemistry 47, 4770-9 (2008).
37. Rye, K. A. & Barter, P. J. The influence of apolipoproteins on the structure and function of spheroidal, reconstituted high density lipoproteins. J Biol Chem 269, 10298-303 (1994).
38. Smith, P. K. et al. Measurement of protein using bicinchoninic acid. Anal Biochem 150, 76-85 (1985).
39. Gaubatz, J. W. et al. Dynamics of dense electronegative low density lipoproteins and their preferential association with lipoprotein phospholipase A(2). J Lipid Res 48, 348-57 (2007).
40. Grigorieff, N. FREALIGN: high-resolution refinement of single particle structures. J Struct Biol 157, 117-25 (2007).
41. Frank, J. et al. SPIDER and WEB: processing and visualization of images in 3D electron microscopy and related fields. J Struct Biol 116, 190-9 (1996).
42. Ludtke, S. J., Baldwin, P. R. & Chiu, W. EMAN: semiautomated software for high-resolution single-particle reconstructions. J Struct Biol 128, 82-97 (1999).
43. Galkin, V. E., Orlova, A., Cherepanova, O., Lebart, M. C. & Egelman, E. H. High-resolution cryo-EM structure of the F-actin-fimbrin/plastin ABD2 complex. Proc Natl Acad Sci USA 105, 1494-8 (2008).
44. Bottcher, B., Wynne, S. A. & Crowther, R. A. Determination of the fold of the core protein of hepatitis B virus by electron cryomicroscopy. Nature 386, 88-91 (1997).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. An antibody or fragment thereof capable of specifically binding to an epitope of Cholesterol Ester Transfer Protein (CETP) having the sequences of loops
44-61
(ITGEKAMMLLGQVKYGLH; SEQ ID NO: 1),
95-116
(GTLKYGYTTAWWLGIDQSIDFE; SEQ ID NO: 2),
151-171
(LLHLQGEREPGWIKQLFTNFI; SEQ ID NO: 3),
288-319
(GRLMLSLMGDEFKAVLETWGFNTNQEIFQEVVGGFPSQA,
SEQ ID NO: 5)
or
349-360
(FLFPRPDQQHSVA; SEQ ID NO: 6);
or
KYGYTTAWWLGIDQS(SEQ ID NO: 4),
or
YTTAWWLGID(SEQ ID NO: 7).

2. The antibody or fragment thereof of claim 1, wherein the epitope is presented by an amino acid sequence selected from the group consisting of, KYGYTTAWWLGIDQS (SEQ ID NO:4), or YTTAWWLGID (SEQ ID NO:7).

3. A polynucleotide encoding the antibody or fragment thereof of claim 1.

4. A vector comprising the polynucleotide of claim 3.

5. A cell comprising the polynucleotide of claim 3 or the vector of claim 4.

6. A hybridoma capable of producing an antibody or fragment thereof of claim 1.

7. A method of isolating a peptide of interest, comprising:
(a) contacting (i) a peptide of interest linked to an amino acid sequence selected from SEQ ID NOS:1-7 or a fragment thereof, and (ii) the antibody or fragment thereof capable of binding to an epitope of SEQ ID NOS:1, 2, 3, 5, or 6, or SEQ ID NOS: 4 or 7, and
(b) separating at least a partial population of the antibody or fragment thereof, and any bound molecule thereto, from molecules not bound to the antibody or fragment thereof.

8. The method of claim 7, wherein the contacting step comprises introducing a first solution comprising the peptide of interest linked to the amino acid sequence selected from SEQ ID NOS:1-7 or fragment thereof, and a second solution comprising the antibody or fragment thereof.

9. The method of claim 8, further comprising linking the peptide of interest to the amino acid sequence selected from SEQ ID NOS:1-7 or a fragment thereof.

10. The method of claim 7, further comprising expressing the peptide of interest linked to the amino acid sequence selected from SEQ ID NOS:1-7 or a fragment thereof in a host cell, or in vitro in a reaction solution, comprising a polynucleotide encoding peptide of interest linked to the amino acid sequence selected from SEQ ID NOS:1-7 or a fragment thereof.

11. The method of claim 7, further comprising linking a first polynucleotide encoding the peptide of interest and second polynucleotide encoding the amino acid sequence selected from SEQ ID NOS:1-7 or a fragment thereof.

12. A kit comprising: a vector comprising a nucleotide sequence encoding the amino acid sequence selected from SEQ ID NOS:1-7 or a fragment thereof linked to one or more restriction sites, and an antibody or fragment thereof capable of specifically binding to an epitope of the amino acid sequence selected from SEQ ID NOS:1-7 or a fragment thereof.

13. An anti-human CETP N-terminus monoclonal antibody or a F(ab′)₂ or Fab′ fragment from said monoclonal antibody, wherein said monoclonal antibody inhibits CETP interaction with lipoproteins and inhibits cholesterol ester transfer activity of human CETP, wherein said monoclonal antibody inhibits CETP interaction with lipoproteins and inhibits cholesterol ester transfer activity of human CETP by binding to one of the CETP N-terminal domain loops 44-61, 95-116, or 151-171 or one of the CETP C-terminal domain loops 288-319 or 349-360.

14. The anti-human CETP C-terminus monoclonal antibody or a F(ab′)₂ or Fab′ fragment from said monoclonal antibody of claim 19, wherein said monoclonal antibody inhibits CETP interaction with lipoproteins and inhibits cholesterol ester transfer activity of human CETP, wherein said monoclonal antibody inhibits CETP interaction with lipoproteins and inhibits cholesterol ester transfer activity of human CETP by binding to one of the CETP C-terminal domain loops 288-319 or 349-360.

15. A recombinant chimeric monoclonal antibody or a F(ab′)₂ or Fab′ fragment from said monoclonal antibody of claim 13, wherein said monoclonal antibody comprises a variable region from the monoclonal antibody of claim 13 and a constant region from a human immunoglobulin.

16. A recombinant humanized monoclonal antibody or a F(ab′)₂ or Fab′ fragment from said monoclonal antibody of claim 13, wherein said monoclonal antibody comprises a part of or the whole of the complementarity determining regions of the hypervariable region from the monoclonal antibody of claim 13, framework regions of the hypervariable region from a human immunoglobulin and a constant region from a human immunoglobulin.

17. An immobilized monoclonal antibody or immobilized antibody fragment which is prepared by immobilizing the monoclonal antibody or F(ab′)₂ or Fab′ fragment of claim 13 on an insoluble carrier.

18. The immobilized monoclonal antibody or immobilized F(ab′)₂ or Fab′ fragment of claim 13 wherein said insoluble carrier is selected from the group consisting of a plate, a test tube, a tube, beads, a ball, a filter and a membrane.

19. The immobilized monoclonal antibody or immobilized F(ab′)₂ or Fab′ fragment of claim 13 wherein said insoluble carrier is one used for affinity purification.

20. A labeled monoclonal antibody or labeled antibody fragment which is prepared by labeling the monoclonal antibody or F(ab′)₂ or Fab′ fragment of claim 13 with a label that provides a detectable signal independently or by reaction with another substance.

21. The labeled monoclonal antibody or labeled F(ab′)₂ or Fab′ fragment of claim 13 wherein said label is selected from the group consisting of an enzyme, a fluorescent material, a chemiluminescent material, biotin, avidin, nanoparticle, and a radioisotope.

22. A kit for immunoassay to detect the N-terminal domain of human CETP comprising the monoclonal antibody or F(ab′)₂ or Fab′ fragment of claim 13.

23. A kit for immunoassay to detect the C-terminal domain of human CETP comprising the monoclonal antibody or F(ab′)₂ or Fab′ fragment of claim 14.

24. A kit for immunoassay to detect the N-terminal domain of human CETP comprising the labeled monoclonal antibody or labeled F(ab′)₂ or Fab′ of claim 13.

25. A kit for immunoassay to detect the C-terminal domain of human CETP comprising the labeled monoclonal antibody or labeled F(ab′)₂ or Fab′ of claim 14.

26. A kit for separation or purification of human CETP comprising the immobilized monoclonal antibody or immobilized F(ab′)₂ or Fab′ fragment of claim 13.

27. A composition comprising the monoclonal antibody or F(ab′)₂ or Fab′ fragment of claim 13; and a pharmaceutically acceptable carrier.

28. A composition comprising the chimeric monoclonal antibody or F(ab′)₂ or Fab′ fragment of claim 15; and a pharmaceutically acceptable carrier.

29. A composition comprising the humanized monoclonal antibody or F(ab′)₂ or Fab′ fragment of claim 16; and a pharmaceutically acceptable carrier.

30. A method for treating or preventing atherosclerosis in a subject comprising administering to said subject an antigenic vaccine peptide comprising a universal helper T cell epitope portion linked to a B cell epitope portion, wherein said B cell epitope portion comprises an epitope of CETP comprising a sequence involved in CETP binding and/or interaction with HDL and found in any of loops 44-61, 95-116, 151-171, 288-319, or 349-360 CETP.

31. The method according to claim 30, wherein said helper T cell epitope portion comprises a helper T cell epitope derived from an antigenic peptide selected from the group consisting of tetanus toxoid, diphtheria toxoid, pertussis vaccine, Bacile Calmette-Guerin (BCG), polio vaccine, measles vaccine, mumps vaccine, rubella vaccine, purified protein derivative of tuberculin, keyhole limpet hemocyanin, hsp70, and combinations thereof.

32. The method according to claim 31, wherein said CETP epitope portion of the antigenic vaccine peptide comprises 7 to 15 consecutive amino acids of amino acids 44-61, 95-11, 151-171, 288-319 and 349-360 of human cholesteryl ester transfer protein (SEQ ID NO:8).

33. The method according to claim 32, wherein the CETP epitope comprising amino acids 98-112 (SEQ ID NO:4) or 101-110 (SEQ ID NO:7) of CETP.

34. The method according to claim 33, wherein the CETP epitope comprising amino acids 101-110 (SEQ ID NO:7) of CETP.

35. The method according to claim 32, wherein the 7 to 15 consecutive amino acids of CETP further comprising one of the following specific epitopes: D42-E46, K56-H60, K94-K98, or D114-E115.

36. The method according to claim 31, wherein the mode of said administration of said antigenic vaccine peptide is selected from the group consisting of intraperitoneal administration, interperitoneal administration, intramuscular injection, intravenous injection, subcutaneous injection, and oral administration.

37. The method according to claim 36, wherein said administration is comprised of one primary dose of said antigenic vaccine peptide followed by one or more booster administrations of said vaccine peptide.

38. The method according to claim 31, wherein said antigenic vaccine peptide is formulated with a pharmaceutically acceptable adjuvant.


SEQUENCE LISTING (8 sequences)

SEQ ID NO: 1 - protein, 18 aa, Artificial sequence
  Human CETP amino acids 44-61 synthetic peptide
ITGEKAMMLLGQVKYGLH

SEQ ID NO: 2 - protein, 22 aa, Artificial sequence
  Human CETP 95-116 amino acid synthetic peptide
GTLKYGYTTAWWLGIDQSIDFE

SEQ ID NO: 3 - protein, 21 aa, Artificial sequence
  Human CETP amino acids 151-171 synthetic peptide
LLHLQGEREPGWIKQLFTNFI

SEQ ID NO: 4 - protein, 15 aa, Artificial sequence
  Human CETP amino acids 98-111 synthetic peptide
KYGYTTAWWLGIDQS

SEQ ID NO: 5 - protein, 39 aa, Artificial sequence
  Human CETP amino acids 288-319 synthetic peptide
GRLMLSLMGDEFKAVLETWGFNTNQEIFQEVVGGFPSQA

SEQ ID NO: 6 - protein, 13 aa, Artificial sequence
  Human CETP amino acids 349-360 synthetic peptide
FLFPRPDQQHSVA

SEQ ID NO: 7 - protein, 10 aa, Artificial sequence
  Human CETP amino acids 101-110 synthetic peptide
YTTAWWLGID

SEQ ID NO: 8 - protein, 493 aa, Homo sapiens [sequence omitted]